(19) United States
(12) Patent Application Publication
(10) Pub. No.: US 2025/0200331 A1
(43) Pub. Date: Jun. 19, 2025

(54) USING LANGUAGE MODELS TO ASSIST TUMOR BOARD DISCUSSION

(71) Applicant: Siemens Healthineers International AG, Steinhausen (CH)

(72) Inventors: Esa KUUSELA, Espoo (FI); Ismo HAUTALA, Espoo (FI)

(73) Assignee: Siemens Healthineers International AG, Steinhausen (CH)

(21) Appl. No.: 18/539,008

(22) Filed: Dec. 13, 2023

(51) Int. Cl. G06N 3/0455; G06N 3/084; G16H 20/40

(52) U.S. Cl. CPC G06N 3/0455; G06N 3/084; G16H 20/40

(57) ABSTRACT

Embodiments described herein provide for implementing a language model in a use-case for Tumor Board meetings for radiotherapy treatment planning (RTTP) efforts and treatment planning assistance, which requires high-levels of accuracy and/or precision in the outputs generated by the LLM and presented to members of the Tumor Board participating in a Tumor Board discussion, which may include live meetings or asynchronous online discussions. Tumor Board Application (TBA) software collects from discussions of a Tumor Board meeting to train the LLM on predicting outputs that contribute information about the patient, proposed RTTP, or aspects of the patient treatment. An AI agent participates in the Tumor Board discussion to ingest the inputs of the members of the Tumor Board and output the responsive text produced by the LLM, thereby allowing the LLM-powered AI-agent to interact with Tumor Board discussions.

### TECHNICAL FIELD

This application relates generally to generating a radiotherapy treatment plan using a machine learning language processing model.

### BACKGROUND

Radiotherapy or radiation therapy (RT) is one of the main modalities used in cancer treatment, and RT treatment planning (RTTP) is a complex process that contains specific guidelines, protocols, and instructions adopted by different medical professionals, such as clinicians, medical device manufacturers, and the like. Typically, the overall treatment strategy, such as selecting which RT modalities should be used, is discussed and decided in a multi-disciplinary Tumor Board meeting, where one or more RT-oncologists are accompanied by surgeons, radiologists, and general physicians, among others. These meetings should thus be as efficient as possible since multiple key-resources of the clinic are participating in the Tumor Board's reviews and decisions.

A Tumor Board typically involves a multidisciplinary group of professionals who review and consider treatment options (e.g., RT, chemotherapy, surgery) and related healthcare efforts, such as follow-ups, continued care, and psychosocial support. It is possible that the patient history is reviewed (including aspects related to the diagnosis, such pathology statement and images). In many cases, the stage of the cancer may be defined by the tumor board. The Tumor Board may review and discuss patient-specific factors (e.g., age, other medical conditions) to determine how such factors could affect the treatment possibilities. In some circumstances, for example, the Tumor Board may determine that the RTTP is an appropriate component of the patient care. The RTTP creation typically involves a collaboration of multiple professionals who collectively function as the Tumor Board who diagnose the patients and develop the RTTP for the patient. Especially important in this process is the interaction between the members of the tumor board, and providers (e.g., radiation oncologist). It is common that after the oncologist has first generated a plan for treatment using such a computer model, the oncologist may perform multiple rounds of additional interaction with the computer model because there were ambiguities in the original request, aspects of the plan needed to be improved, or the oncologist wanted to verify that better plans are not available. The information discussed by the members of the tumor board is typically memorialized as the RTTP or tasks of the RTTP.

The discussions by the tumor board meetings, however, often contain useful information that could be cross-referenced and verified to further develop the treatment options (e.g., RTTP options for RT; chemotherapy options and planning; surgery options and planning; types of follow-up care) or tasks, though this information is rarely captured and analyzed in a manner that could benefit downstream computing processes or medical tasks.

### SUMMARY

Embodiments disclosed herein include computing systems that execute software components for machine-learning architecture functions, including an artificial intelligent assistant, to improve information capture and analysis for Tumor Board meetings to improve the efficiency and efficacy of Tumor Board efforts to evaluate patient data and patient attributes and develop an RTTP (or other types of treatment options) for the patient. A computing system includes hardware and software components for an artificial intelligence (AI) productivity assistance (“AI assistant” or “AI agent”) program or computing platform powered by one or more models, such as Large Language Models (LLMs) trained to identify and show relevant knowledge to synchronous or semi-synchronous discussions (e.g., live meetings, live teleconferences, chat windows) or asynchronous discussions (e.g., online posts). It should be appreciated that embodiments described herein mention an RTTP for RT-based treatment options, but embodiments are not so limited. Embodiments may include applying the AI assistant and LLMs on the information created for and by a collaborative group of professionals participating in a Tumor Board.

There are currently multiple vendors providing access to pre-trained LLMs. The latest LLMs have demonstrated a remarkable capability to continue contributing to, enriching, or otherwise facilitating natural language feeds (e.g., answering questions, and generating text-based summaries of articles). A shortcoming of current LLMs includes deficient or low levels of self-awareness of the accuracy or correctness of the responses. Many LLMs often have a tendency of “hallucinating” content if the continuation of a given stream of text (sometimes referred to as “tokens”) or other inputs requires the LLM to do so. This is making direct usage of LLMs impractical in situations requiring high accuracy and correctness of the information of the generated text or other types of outputs. Embodiments described herein provide for implementing an LLM in a use-case for Tumor Board meetings for RTTP efforts and treatment planning assistance, which requires high levels of accuracy and/or precision in the outputs generated by the LLM and presented to members of the Tumor Board participating in a Tumor Board discussion, which may include live meetings or asynchronous online discussions.

In order to facilitate the use of LLM in a Tumor Board meeting, embodiments provide for a collaborative Tumor Board Application (TBA), which may be a stand-alone application or plug-in to another application. The TBA is configured to collect data for, and from, a discussion in a Tumor Board meeting, and allow the LLM-powered AI agent to interact with Tumor Board discussions. The data may be collected in the form of various input modalities, such as text entered by end-users (participating members) into the TBA software or speech audio signals captured at the microphones of a conference room or user devices and converted into text for the TBA software. The LLM is trained to predict a responsive output or determine a request for more information about the patient and generate the responsive output text to continue the ongoing discussion of the Tumor Board members. The responsive output text (or other forms of predicted output data) is presented in the discussion interface by a corresponding AI agent, which is trained and configured to participate in the discussion as a participant of the Tumor Board discussion or to otherwise contribute to the information available to the Tumor Board members and the computing devices of Tumor Board members.

In an embodiment, a method comprises presenting, by a processor, a user interface providing an interaction interface between a plurality of medical professionals communicating regarding a radiation therapy treatment of a patient during a tumor board review; receiving, by the processor from the interaction interface, a first input comprising a first patient attribute of the patient and a second input corresponding to the radiation therapy treatment of the patient; executing, by the processor, a machine learning language processing model using the first input and the second input to predict a treatment attribute for the patient, the machine learning language processing model configured to identify a second radiation therapy treatment for a second patient that corresponds to the first input and the second input based on a tumor board review for the second patient to predict the treatment attribute for the patient, wherein the machine learning language processing model is trained using a set of transcriptions of a set of tumor board reviews for a set of previously implemented radiation therapy treatments; presenting, by the processor on the interaction interface, the treatment attribute for the patient; and in response to receiving an indication of approval, transmitting, by the processor, the treatment attribute, the first input, and the second input to a radiation therapy plan optimizer as an instruction to generate a radiotherapy treatment plan for the patient.

The treatment attribute for the patient may be a timeline of radiation therapy treatment of the patient.

The method may include presenting, by the processor on the interaction interface, at least one of a medical image, laboratory data, or test result of the patient.

The method may include presenting, by the processor on the interaction interface, a hyperlink configured to direct the interaction interface to third-party data associated with the radiation therapy treatment.

The machine learning language processing model may be further trained using previously performed radiation therapy treatments.

The method may include, in response to the second input satisfying a predetermined threshold, presenting, by the processor in the interaction interface, a warning message.

The first input may be a medical image.

In another embodiment, a system comprises a server comprising a processor and a non-transitory computer-readable medium containing instructions. When executed by the processor, the instructions causes the processor to: present a user interface providing an interaction interface between a plurality of medical professionals communicating regarding a radiation therapy treatment of a patient during a tumor board review; receive, from the interaction interface, a first input comprising a first patient attribute of the patient and a second input corresponding to the radiation therapy treatment of the patient; execute a machine learning language processing model using the first input and the second input to predict a treatment attribute for the patient, the machine learning language processing model configured to identify a second radiation therapy treatment for a second patient that corresponds to the first input and the second input based on a tumor board review for the second patient to predict the treatment attribute for the patient, wherein the machine learning language processing model is trained using a set of transcriptions of a set of tumor board reviews for a set of previously implemented radiation therapy treatments; present, on the interaction interface, the treatment attribute for the patient; and in response to receiving an indication of approval, transmit the treatment attribute, the first input, and the second input to a radiation therapy plan optimizer as an instruction to generate a radiotherapy treatment plan for the patient.

The treatment attribute for the patient may be a timeline of radiation therapy treatment of the patient.

The instructions may further cause the processor to present, on the interaction interface, at least one of a medical image, laboratory data, or test result of the patient.

The instructions may further cause the processor to present, on the interaction interface, a hyperlink configured to direct the interaction interface to third-party data associated with the radiation therapy treatment.

The machine learning language processing model may be further trained using previously performed radiation therapy treatments.

The instructions may further cause the processor to, in response to the second input satisfying a predetermined threshold, presenting, by the processor in the interaction interface, a warning message.

The first input may be a medical image.

In yet another embodiment, a system comprises a computer configured to display a user interface; and a server in communication with the computer. The server is configured to: present the user interface providing an interaction interface between a plurality of medical professionals communicating regarding a radiation therapy treatment of a patient during a tumor board review; receive, from the interaction interface, a first input comprising a first patient attribute of the patient and a second input corresponding to the radiation therapy treatment of the patient; execute a machine learning language processing model using the first input and the second input to predict a treatment attribute for the patient, the machine learning language processing model configured to identify a second radiation therapy treatment for a second patient that corresponds to the first input and the second input based on a tumor board review for the second patient to predict the treatment attribute for the patient, wherein the machine learning language processing model is trained using a set of transcriptions of a set of tumor board reviews for a set of previously implemented radiation therapy treatments; present, on the interaction interface, the treatment attribute for the patient; and in response to receiving an indication of approval, transmit the treatment attribute, the first input, and the second input to a radiation therapy plan optimizer as an instruction to generate a radiotherapy treatment plan for the patient.

The treatment attribute for the patient may be a timeline of radiation therapy treatment of the patient.

The server may be further configured to present, on the interaction interface, at least one of a medical image, laboratory data, or test result of the patient.

The server may be further configured to present, on the interaction interface, a hyperlink configured to direct the interaction interface to third-party data associated with the radiation therapy treatment.

The machine learning language processing model may be further trained using previously performed radiation therapy treatments.

The server may be further configured to, in response to the second input satisfying a predetermined threshold, presenting, by the processor in the interaction interface, a warning message.

### BRIEF DESCRIPTION OF THE DRAWINGS

Non-limiting embodiments of the present disclosure are described by way of example with reference to the accompanying figures, which are schematic and are not intended to be drawn to scale. Unless indicated as representing the background art, the figures represent aspects of the disclosure.

FIG. 1 illustrates components of a system for conducting Tumor Board discussions and developing RTTPs using machine-learning language model processing, according to an embodiment.

FIG. 2 shows an operational workflow of a method performed in hardware and software computing components that host and execute a machine-learning language-processing, in accordance with an embodiment.

FIG. 3 depicts an example user interface of collaboration software for member-users of a Tumor Board discussion to collaborate and interact with machine learning language models, in accordance with an embodiment.

FIG. 4 illustrates a dataflow amongst components of a system hosting a TBA for Tumor Board collaboration and RTTP, according to an embodiment.

FIG. 5 depicts an LLM-prompt interface displaying a template of a LLM prompt to generate predicted outputs for contributing to and continuing a discussion of a Tumor Board, according to an embodiment.

### DETAILED DESCRIPTION

Reference will now be made to the illustrative embodiments depicted in the drawings, and specific language will be used here to describe the same. It will nevertheless be understood that no limitation of the scope of the claims or this disclosure is thereby intended. Alterations and further modifications of the inventive features illustrated herein, and additional applications of the principles of the subject matter illustrated herein, which would occur to one skilled in the relevant art and having possession of this disclosure, are to be considered within the scope of the subject matter disclosed herein. Other embodiments may be used and/or other changes may be made without departing from the spirit or scope of the present disclosure. The illustrative embodiments described in the detailed description are not meant to be limiting of the subject matter presented.

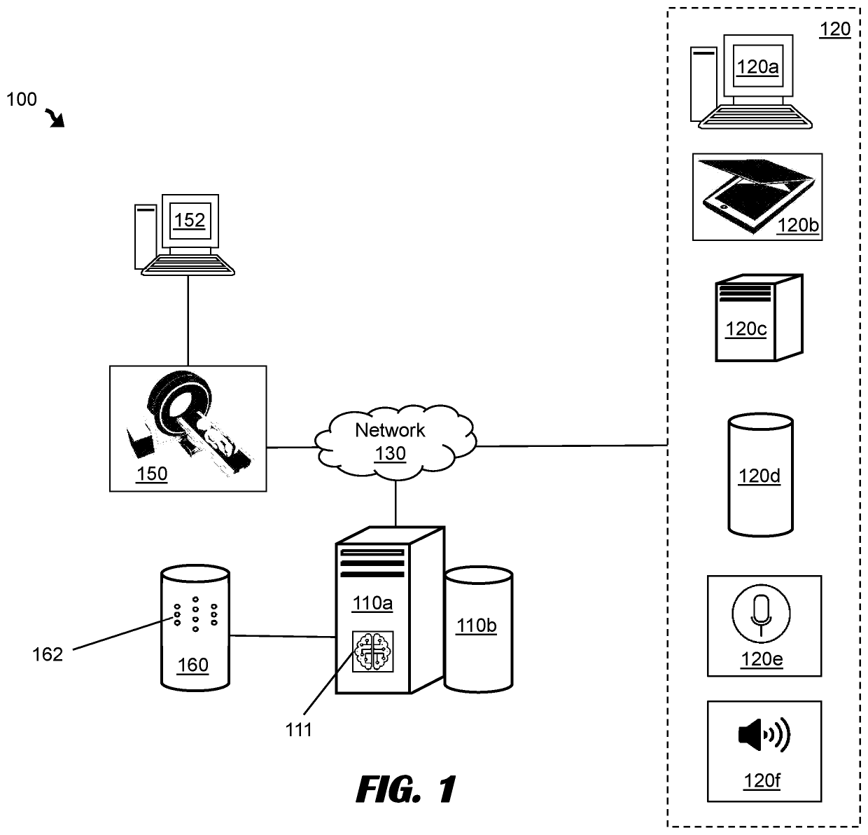

FIG. 1 illustrates components of a system 100 for conducting Tumor Board discussions and developing RTTPs using machine-learning language model processing, according to an embodiment. The system 100 may include an analytics server 110a, a system database 110b, a machine learning language processing model 111 for presenting contextual information to participants of a Tumor Board discussion, end-user devices 120a-120f (collectively end-user devices 120), a medical device 150, a medical device computer 152, a database 160, and a radiotherapy plan optimizer 162. Various components depicted in FIG. 1 may belong to a radiation therapy treatment clinic at which patients may receive radiation therapy treatment, in some cases via one or more radiation therapy machines (e.g., the medical device 150).

The system 100 is not confined to the components described herein and may include additional or other components, not shown for brevity, which are to be considered within the scope of the embodiments described herein.

The above-mentioned components may be connected to each other through one or more networks 130. Examples of the network 130 may include, but are not limited to, private or public local-area networks (LAN), wireless local-area networks (WLAN), metropolitan-area networks (MAN), wide-area networks (WAN), and the Internet. The network 130 may include wired and/or wireless communications according to one or more standards and/or via one or more transport mediums. The communication over the network 130 may be performed in accordance with various communication protocols such as Transmission Control Protocol and Internet Protocol (TCP/IP), User Datagram Protocol (UDP), and IEEE communication protocols. In one example, the network 130 may include wireless communications according to Bluetooth specification sets or another standard or proprietary wireless communication protocol. In another example, the network 130 may also include communications over a cellular network, including, e.g., a GSM (Global System for Mobile Communications), CDMA (Code Division Multiple Access), or EDGE (Enhanced Data for Global Evolution) network.

The analytics server 110a may generate and display an electronic platform configured to interface a user with the machine learning language processing model 111 and for receiving patient information and outputting the results of execution of the machine learning language processing model 111 and the radiotherapy plan optimizer 162. The electronic platform may include graphical user interfaces (GUI) displayed on each of the end-user devices 120, the medical device 150, and/or the medical device computer 152. An example of the electronic platform generated and hosted by the analytics server 110a may be a web-based application or a website configured to be displayed on different electronic devices, such as mobile devices, tablets, personal computers, and the like.

The platform hosted on the analytics server 110a or other device of the system 100 includes collaboration software accessible to the user devices 120 of the participating members of the Tumor Board. The collaboration software may include any type of software facilitating user-group collaborations, which may include live interaction software (e.g., teleconferencing software) or asynchronous collaborations (e.g., online postings). Non-limiting examples of the collaboration software may include MS Teams®, Skype®, WebEx®, Slack®, and Twilio®, among others. The analytics server 102 may execute a Tumor Board Application (TBA) that comprises, invokes, executes, and manages the operations of the AI agent and machine learning language processing model 111, among other functions. The TBA collects and ingests various types of inputs and feeds the inputs into the AI agent and machine learning language processing model 111.

In some embodiments, the TBA (including the AI agent and machine learning language processing model 111) is a software module component (e.g., plug-in) of the collaboration software of the platform of the analytics server 110a. In some embodiments, the collaboration software makes calls to the TBA software of the analytics server 110a to provide inputs to, and invoke operations of, the AI agent and machine learning language processing model 111.

The information displayed by the TBA of the electronic platform can include, for example, input elements to receive data associated with a patient to be treated (e.g., plan objectives) and display results of predictions for AI-generated text for continuing the Tumor Board discussion, as produced by the machine learning language processing model 111, which may include various formats of responsive predicted outputs (e.g., text, images, or videos generated in response to inputs received through the TBA or electronic platform). Optionally, the outputs produced by the machine learning language processing model 111 of the TBA may be fed to the radiotherapy plan optimizer 162 (e.g., a predicted radiotherapy plan). The analytics server 110a may then display the results for the participants of the Tumor Board at a user device 120, and/or other medical professional at the medical device 150. In some embodiments, the medical device 150 can be a diagnostic imaging device or a treatment delivery device.

The analytics server 110a may be any computing device comprising a processor and non-transitory machine-readable storage capable of executing the various tasks and processes described herein. The analytics server 110a may employ various processors such as central processing units (CPU) and graphics processing unit (GPU), among others. Non-limiting examples of such computing devices may include workstation computers, laptop computers, server computers, and the like. While the system 100 includes a single analytics server 110a, the analytics server 110a may include any number of computing devices operating in a distributed computing environment, such as a cloud environment.

End-user devices 120 of a tumor board or physicians may be any computing device comprising a processor and a non-transitory machine-readable storage medium capable of performing the various tasks and processes described herein. Non-limiting examples of an end-user device 120 may be a workstation computer, laptop computer, tablet computer, and server computer. In operation, various users may use end-user devices 120 to access the GUI operationally managed by the analytics server 110a. Specifically, the end-user devices 120 may include a clinic computer 120a, a clinic server 120b, and medical professional devices 120c, which may include any electronic devices operated by members of the Tumor Board, medical professionals, and scientists that access and review various types of patient-related treatment data and RTTPs for the patient, among other types of data and information exchanges. For instance, members of the Tumor Board may operate the medical professional devices 120c to review patient-related treatment data to develop consensus of a diagnosis and RTTP for the patient. Even though referred to herein as “end-user” devices, these devices may not always be operated by end-users. For instance, the clinic server 120b may not be directly used by an end-user. However, the results stored on the clinic server 120b may be used to populate various GUIs accessed by an end-user via the medical professional device 120c. Patient-related information generated by the various types of devices of the system 100, outside the context of Tumor Board discussions may be stored into the system database 100b. The stored patient data may be referenced by the TBA during Tumor Board discussions and/or referenced by the TBA for training the machine learning language processing model 111 or the AI agents.

The medical device 150 may be a radiation therapy machine configured to implement a patient's radiotherapy treatment. The medical device 150 may also be in communication with a medical device computer 152 that is configured to display various GUIs discussed herein. For instance, the analytics server 110a may display the results predicted by the radiotherapy plan optimizer 162 onto the computing devices described herein.

The machine learning language processing model 111 may be stored in the system database 110b. The machine learning language processing model 111 may be configured or trained to automatically generate text, image, or video responses based on inputs received at a user interface or other types of inputs (e.g., speech captured at a conference room microphone or microphone of an end-user device 120). The machine learning language processing model 111 can be configured and trained to receive the various inputs from the members of the Tumor Board and patient attributes for a patient as input and automatically generate various predicted responsive outputs for continuing the discussion.

The user-provided inputs include, for example, text inputs entered by members of the Tumor Board via user interfaces, or audio signals containing speech audio of the members of the Tumor Board captured by microphones of the user devices 120 or conference room. The TBA may include Automated Speech Recognition (ASR) software that converts speech audio to written text. The ASR software comprises a machine-learning architecture trained to detect portions or frames of the audio signal containing the speech audio of a member-speaker. The ASR also comprises and applies Natural Language Processing (NLP) layers of the machine-learning architecture that generates text-based output from the portions of the audio signal containing the detected speech audio. The text generated by the ASR may be fed as an input to the machine learning language processing model 111. The machine learning language processing model 111 is trained to simulate and contextually continue a conversation with the Tumor Board, based upon the text of the Tumor Board discussion and the patient-related data indicating the patient attributes over time.

In some embodiments, the analytics server 110a may execute a radiotherapy plan optimizer 162 to generate one or more treatment attributes for an RTTP complying with any radiation therapy plan objectives based on patient attributes of a patient for which the radiotherapy treatment plan is being generated. The radiotherapy plan optimizer 162 can be stored in the database 160. The radiotherapy plan optimizer 162 can generate the one or more treatment attributes, for example, by iteratively calculating the one or more treatment attributes where, with each iteration, the radiotherapy plan optimizer 162 can revise the one or more treatment attributes of the RTTP in accordance with a cost value. The analytics server 110a may deploy the radiotherapy plan optimizer 162 to generate an RTTP for a patient based on patient attributes for the patient. The radiotherapy plan optimizer 162 may iteratively calculate one or more treatment attributes of the RTTP. For instance, with each iteration, the radiotherapy plan optimizer 162 may generate a candidate RTTP having various attributes. The plan optimizer 162 may then use one or more loss functions to calculate a cost value for the generated candidate RTTP. The cost value may indicate a likelihood of the candidate RTTP violating a set of rules, whether internal and/or external rules. For instance, the cost value may indicate whether the candidate RTTP violates any of the plan objectives. The radiotherapy plan optimizer 162 may analyze the cost value. If needed (e.g., when the cost value satisfies a threshold), the radiotherapy plan optimizer 162 may revise the candidate RTTP and re-execute its loss function to generate a new cost value. Depending on whether the new cost function is increasing or decreasing, the plan optimizer computer model may revise the candidate RTTP again and recalculate the cost value. The radiotherapy plan optimizer 162 may continue this iterative approach until converging upon an RTTP (or the final RTTP) that has a cost value that satisfies a threshold. In some implementations, the treatment attribute for the patient may also indicate how the radiotherapy treatment may be combined, or sequentially implemented, with other types of treatment modalities (e.g., surgery, chemotherapy).

The Tumor Board may implement the features and functions of the analytics server 110a described herein for discussing potential, ongoing, or prior radiotherapy-based treatments, but embodiments are not so limited to discussions for potential radiotherapy treatments. For instance, the Tumor Board may implement and benefit from the LLM and AI-related features of the system 100 when the Tumor Board is discussing any number of potential treatment options (e.g., surgery, radiotherapy treatment, chemotherapy) and then ultimately decides that another type of treatment, aside from a radiotherapy treatment, should be used to treat the patient. In some circumstances, for example, the analytics server 110a or other features of the system 100 may generate various outputs about the treatment options discussed by the Tumor Board that ultimately dissuade the Tumor Board from pursuing radiotherapy.

The analytics server 110a can identify the treatment attributes that the radiotherapy plan optimizer 162 determined have a cost value that satisfies the threshold. The analytics server 110a can present the treatment attributes as an RTTP at the end-user device 120 being accessed by the user generating the radiotherapy treatment plan. The user can implement the RTTP or the analytics server 110a or the end-user device 120 can use the RTTP to automatically control the medical device 150 based on attributes of the RTTP to treat the patient.

The system database 110b may contain data needed to train the machine learning language processing model 111. For instance, the system database 110b may include data associated with previously treated patients, such as patient diagnosis data (e.g., tumor data or tumor location), biometric data (e.g., BMI, body weight, height, or various other bodily measurements), and the like. Additionally, the system database 110b may include tumor board discussions (audio, transcription, and/or video recording of tumor board meetings) corresponding to the previously treated patients as well. Therefore, the system database 110b may include all data associated with how the previously treated patients were diagnosed and treated. As described herein, the analytics server 110a may use the data stored within the system database 110b to train the machine learning language processing model 111.

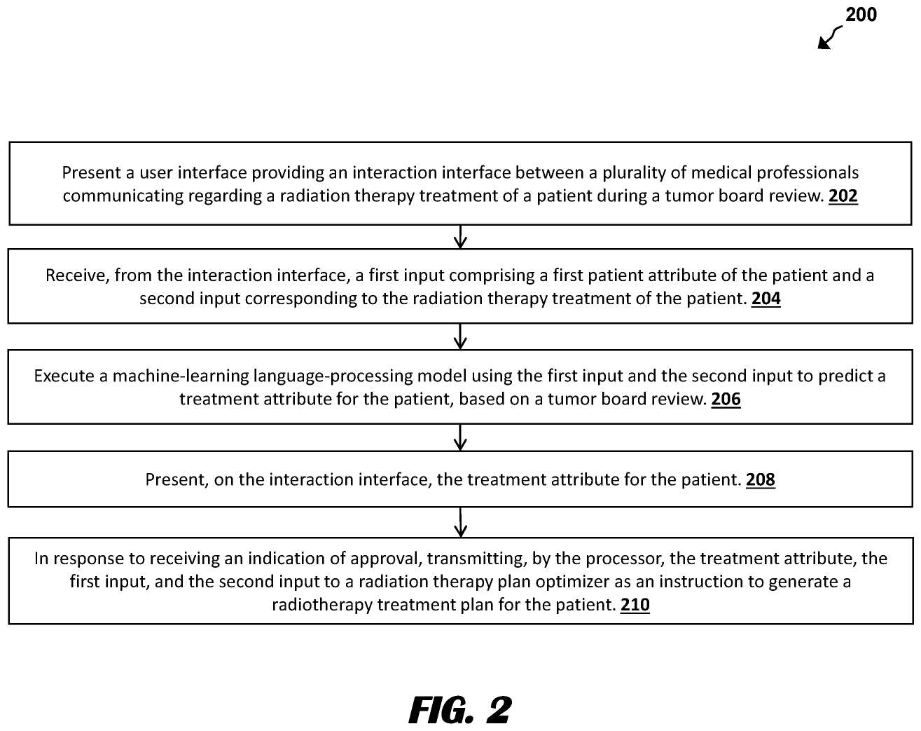

FIG. 2 shows an operational workflow of a method 200 performed in hardware and software computing components that host and execute a machine-learning language processing, in accordance with an embodiment. The method 200 may include steps 202-212. However, other embodiments may include additional or alternative steps or may omit one or more steps altogether. The method 200 is described as being executed by a server, such as the analytics server described in FIG. 1. However, one or more steps of the method 200 may be executed by any number of computing devices operating in the distributed computing system described in FIG. 1. For instance, one or more computing devices may locally perform some or all of the steps described in FIG. 2.

Using the method 200, the analytics server can implement a combination of a machine learning language processing model of a TBA for predicting responsive text-based outputs or other types of responsive outputs that contribute to Tumor Board discussions focused on developing an RTTP. To do so, the TBA can host or provide an interaction interface (e.g., conference audiovisual inputs and outputs; a text-based collaboration interface) for capturing inputs from a conference room microphone or a computing device being accessed by a user (e.g., member-user of the Tumor Board). In some cases, the analytics server hosting the TBA retrieves patient-related data from a patient database. Through the interaction interface or by capturing various types of user inputs (e.g., microphone capturing voice speech of Tumor Board members speaking during Tumor Board meetings), the users can input patient attributes of a patient or any other data or the TBA may query the patient-related data from the patient database. The analytics server can receive the inputs and provide the inputs to the machine learning language processing model. The machine learning language processing model of the TBA can process the inputs and generate responses (e.g., text, images, video, etc.) that continue and enrich the TBA discussions based on the inputs provided by the members of the Tumor Board or retrieved from the patient database.

At step 202, the analytics server may present a user interface on a computing device. The analytics server may generate the user interface of the TBA (or collaboration software having the TBA) and transmit the user interface to the computing device over a network. The analytics server may generate and transmit the user interface to the computing device when providing an electronic platform that facilitates Tumor Board discussions regarding treatment considerations and planning (e.g., RTTP) for patients based on inputs from the members of the Tumor Board.

The analytics server can generate the user interface to have an interaction interface. The interaction interface can be an interface that enables the communication between a user viewing and providing inputs into an interaction interface and the analytics server or a machine learning language processing model. The machine learning language processing model can be a large language model that has been trained to generate text, image, and/or video responses to text, image, and/or video input. The machine learning language processing model can be or include a neural network, such as a neural network with a transformer architecture. The interaction interface can enable a form of communication similar to a conversation between two humans that can involve back-and-forth exchanges of messages between participants in the Tumor Board discussion. In this case, the participants may be expert-users who are members of the Tumor Board, such as an oncologist or other clinician, and an AI agent that implements the machine learning language processing model to ingest inputs from the discussion and provide the outputs of the machine learning language processing model to the Tumor Board interface.

In some implementations, the interaction interface can simulate an instant messaging application conversation between the experts and the machine learning language processing model. For example, a user can input text into the interaction interface. The interaction interface can be a feed (e.g., a user feed). The text can be or include patient attributes for a patient receiving radiotherapy treatment. For example, the user can select a send button to cause the text to be transmitted to the analytics server. The analytics server can input the text including the patient attributes into the machine-learning language processing model and execute the machine-learning language processing model. Based on the input, the language machine learning model may output a response in text or additional types of information, such as media image data containing image scans, charts/graphs, and the like. The analytics server may present the response in the interaction interface. For instance, the response text may be presented in the interaction interface below the most recent input text by the user, thereby displaying the inputs and responses as a running “chat” interaction within in the interaction interface. The user can respond to the response from the machine learning language processing model with text following the response and submit the user input response. This process can repeat any number of times to simulate an instant message application conversation.

The interaction interface can cover a portion of the user interface of the collaboration software or TBA or be a widget of the user interface of the collaboration software or TBA. The user interface can include other portions or widgets that offer differing functionality, such as external software tools. While the interaction interface can enable communication between the Tumor Board participants and the AI agent(s) implementing corresponding machine learning language processing model(s), the analytics server can also include a portion of the user interface that displays, for example, different patient attributes of patients, treatment options mentioned in the Tumor Board discussion, or other types of outputs generated by a machine learning language processing model. For example, the TBA may capture an audio signal in which a participating member mentions a particular patient attribute, treatment option, or other aspect of the patient's treatment planning, and convert the audio signal into a text input for the AI agent. The machine learning language processing model and the AI agent may be trained to, for example, provide additional information about the particular patient attribute, treatment option, or other aspect of the patient's treatment planning mentioned by the participating member. The AI agent may include this additional information as an output to the user interface presenting the ongoing Tumor Board discussion.

The TBA may receive an input indicating an identifier (e.g., a name) of the patient into the user interface or the platform provided by the analytics server. Responsive to receiving the input, the analytics server can retrieve patient data (e.g., one or more patient attributes) regarding the patient from non-transitory memory containing database records of the patient database. Examples of patient attributes the analytics server may retrieve include computed tomography (CT) scans of the patient or a tumor of the patient, images of the patient or a tumor of the patient, previously collected patient attributes of the patient, such as data collected from previous health tests, among others. The analytics server can present the retrieved patient data on the user interface in another widget or a portion of the user interface separate or adjacent to the interaction interface. In some cases, the portion or widget of the user interface including the retrieved patient data can be separated from the interaction interface on the user interface by a line (e.g., a vertical line going across the width or length of the user interface (e.g., along the x-axis or the y-axis) of the user interface).

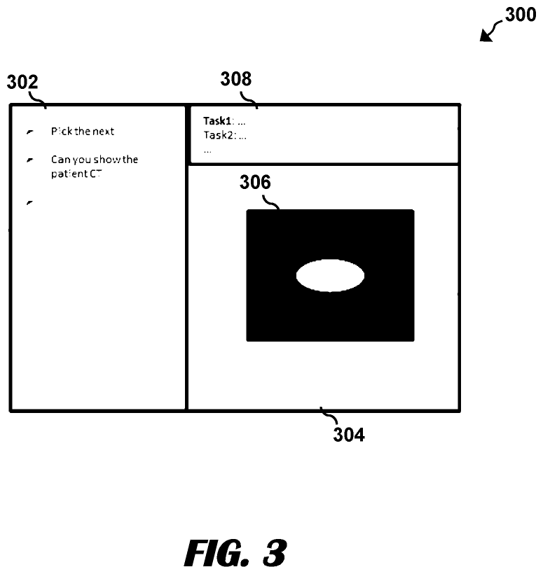

In a non-limiting example, referring now to FIG. 3, the analytics server can generate a user interface 300 to include an interaction interface 302 and a patient data interface 304 of a platform hosting collaboration software and a TBA for contributing to Tumor Board discussions. The interaction interface 302 can be a portion of the user interface 300 that users (e.g., members of a Tumor Board participating in the discussion) can operate or access to interact with a machine learning language processing model. The user or the TBA can input text, images, and/or video into the interaction interface 302 and receive responses from the machine learning language processing model in the interaction interface 302. The patient data interface 304 can be an area of the user interface 300 through which the analytics server (e.g., AI agent and machine learning language processing model) can present retrieved patient data 306 for a patient. The patient data interface 304 can be configured to present any type of data, such as text, images, or videos, which may include various types data relevant to the Tumor Board discussions. The analytics server can generate the user interface 300 and transmit the user interface to a computing device accessed by a user. In some cases, the interaction interface 302 may present data feed containing AI-originated text or data. Additionally or alternatively, the interaction interface 302 may present the data feed having text or other data of corresponding human response or request inputs.

The user interface can include an auxiliary interface 308 to illustrate additional information relevant to the conversation on the interaction interface 302 and/or for the retrieved data on the patient data interface 304 to the Tumor Board members using the user interface 300. The auxiliary interface 308 can include a list of tasks, such as the current task. The tasks can indicate tasks for the user to complete by communicating with the machine learning language processing model. For example, the user can input an option to select a task to generate a RTTP for patient A. Responsive to the input, the machine learning language processing model can identify the input and use the input to determine a response to collect patient data regarding patient A. Another example of a task is to show a CT scan for a patient in the patient data interface 304. Responsive to an input identifying such a task, the machine learning language processing model can identify the input, and the analytics server can retrieve the CT scan for the patient and display the patient scan on the patient data interface 304.

Referring again to FIG. 2, at step 204, the analytics server may receive, from the interaction interface, a first input comprising a first patient attribute of the patient and a second input corresponding to the radiation therapy treatment of the patient. The analytics server can receive the first input from the interaction interface of the user interface. The first input can include a first patient attribute of a patient. The first input can include the patient attribute and any number of other patient attributes. The first patient attribute and the other patient attributes can be any type of patient attribute, such as, for example, height, gender, weight, treatment options for the patient, treatment attributes (e.g., gantry movements, gantry positions, etc.), treatment objectives, attributes regarding a tumor (e.g., size or shape), images of the patient or tumor, tumor stage, the primary site of treatment, endpoints, whether the tumor has been extended, body mass index, blood pressure, medical history (e.g., previous medical treatments received by the patient, etc.). A user can provide the first input and select a send or submit button on the user interface, for example. The analytics server can receive the first input from the computing device presenting the user interface.

In one example, the user can provide the first input by typing the first patient attribute and any other patient attributes of the first input into the interaction interface. For example, the user can type the current height, age, weight, and blood pressure into the interaction interface. Typing the patient attributes into the interaction interface can cause text to appear on the interaction interface. The user can select a submit button responsive to typing the first input into the interaction interface to submit the first input to the analytics server.

In another example, the user can provide the first input by selecting the first attribute and any other patient attributes from patient data that is presented on the user interface. For example, the user can select an image of a tumor of the patient and/or one or more other patient attributes from the patient data. The user can select an option to move the selected patient attributes into the interaction interface or otherwise drag the selected patient attributes into the interaction interface. Upon moving the selected patient attributes into the interaction interface, the user can select an option to submit or send the selected patient attributes to the analytics server. In some cases, the user can select an option to submit or send the selected patient attributes without moving the selected patient attributes to the interaction interface.

In some cases, the user can provide the first input by typing the one or more patient attributes into the interaction interface and selecting one or more patient attributes from the patient data for the patient displayed on the user interface. The user can type one or more patient attributes into the interaction interface, and select one or more patient attributes from the patient data, and select a submit button to transmit the first input to the analytics server.

Referring again to FIG. 3, in a non-limiting example, the user can provide the first input into the interaction interface 302. The user can do so, for example, by typing the first patient attribute and any number of other patient attributes into the interaction interface 302. In some cases, in addition to or instead of typing the first patient attribute into the interaction interface 302, the user can select the first patient attribute and/or any number of other patient attributes from the patient data interface 304. The user can select a submit button on the user interface 300 or otherwise submit the patient attributes by selecting an input/output button to transmit the first patient attribute and any other patient attributes to the analytics server.

Referring again to FIG. 2, at step 206, the analytics server can execute the machine learning language processing model using the first input and the second input to predict a treatment attribute for the patient, based on a tumor board review. The machine learning language processing model may be configured to query and identify a second radiation therapy treatment for a second patient that corresponds to the first input and the second input, based on the Tumor Board's review for the second patient. The machine learning language processing model is trained to predict the treatment attribute for the patient. The machine learning language processing model is trained on a set of transcriptions (sometimes referred to as “discussion logs”) of a set of Tumor Board review discussions, stored in one or more databases, for a set of previously implemented RTTPs for other patients. The analytics server can execute the machine learning language processing model using the first input (e.g., the first patient attribute of the patient and/or any other patient attributes of the first input) as input into the machine learning language processing model.

The machine learning language processing model can generate a response based on the execution. The response can be or include text. The text can be a string of characters or words predicting one or more treatment attributes or RTTPs, providing additional information about aspects of the patient's cases, or requesting a second patient attribute of the patient, among other types of information. The machine learning language processing model can generate the response by implementing learned weights and/or parameters on the data of the first input.

In some cases, the analytics server can include patient data that was stored in memory in the input to the machine learning language processing model with the first input. For example, the analytics server can receive an input identifier (e.g., a name) of the patient from the computing device. The analytics server can query the memory of the patient database, based on the identifier to retrieve patient data regarding the patient from the memory. The retrieved patient data can include one or more patient attributes of the patient. The analytics server can additionally, in some cases, retrieve treatment protocols that are available to use to treat the patient. The analytics server can identify the one or more patient attributes and/or the treatment protocols retrieved from memory and input the one or more patient attributes and/or the treatment protocols into the machine learning language processing model instead of or in addition to the first input received from the user interface. The machine learning language processing model can determine the response based on the first input (e.g., the first patient attribute and any other patient attributes) and the retrieved patient data.

The machine learning language processing model can use one or more templates to determine the response to the first input. In some cases, one or more of the templates can include a list of types of patient attributes. The patient attributes can include data or characteristics for the patient and/or methods of treatment (e.g., one template can correspond to one method of treatment and another template can correspond to another method of treatment). The machine learning language processing model can compare the patient attribute or patient attributes of the first input and, in some cases, any retrieved stored data regarding the patient or available treatment protocols, from memory with the patient attribute or patient attributes and/or ways of treatment of the templates. Based on the comparison, the machine learning language processing model can determine that one or more patient attributes are required to determine an RTTP for the patient based on a template containing required patient attributes and inputs captured from the prior and ongoing Tumor Board discussion.

The templates can correspond to one or more template conditions. The template conditions can be rules that, upon being satisfied, indicate that the template is satisfied and that there is enough data to generate a radiotherapy treatment plant for a patient. In one example, a template condition for a template can indicate that the template is satisfied responsive to the machine learning language processing model receiving data for the patient of each patient attribute type of the template as input. In another example, a template can correspond to one or more template conditions that each indicate a different set of specific patient attributes. One of such template conditions can be satisfied responsive to the machine learning language processing model receiving data for the patient for each patient attribute type of the template condition.

In one non-limiting example, a template can include a list of patient attribute types that includes a gender, a height range, a tumor size, a tumor location, and chemotherapy treatment availability. The template can include a condition that indicates that the machine learning language processing model must receive data for each of the patient attribute types on the list for the condition to be satisfied. The template can include a condition that indicates that the machine learning language processing model must receive data for gender, height range, and the radiotherapy treatment availability for the condition to be satisfied. The template can include conditions that are satisfied based on any permutation or combination of the patient attribute types on the list.

In some cases, the templates can correspond to specific patient attributes. For example, instead of or in addition to having a list of patient attribute types, a template can include a list of specific patient attributes. An example of a list of specific patient attributes is as follows: male, a tumor on the stomach, the patient has a tumor with a five-inch diameter. Lists of specific patient attributes can include any patient attributes and any number of patient attributes. Templates with a list of specific patient attributes instead of a list of patient attribute types can include conditions similar to the conditions described above except the conditions are satisfied based on whether the machine learning language processing model includes the specific patient attributes of the conditions instead of the patient types of the conditions.

In some cases, the templates can correspond to both specific patient attributes and patient attribute types. For example, a template can include both a list of specific patient attributes and a list of patient attribute types. Templates with both a list of specific patient attributes and a list of patient attribute types can include one or more conditions similar to the conditions described above except the conditions can be satisfied based on whether the machine learning language processing model includes both specific patient attributes and patient attribute types.

The machine learning language processing model can receive the first input and retrieve one or more templates from memory. The machine learning language processing model can compare the patient attributes of the first input to the one or more templates. In some cases, based on the comparison, the machine learning language processing model can identify one or more patient attributes or types of patient attributes (e.g., a first type of patient attribute) of a template that is missing from the first input and that would satisfy a condition if a value of the one or more patient attributes or types of patient attributes were to be in the first input and/or the retrieved patient data for the patient. The machine learning language processing model can generate a response with a question or command for the user accessing the interaction interface to input a value for the requested one or more missing patient attributes. Additionally or alternatively, based on the comparison, the machine learning language processing model can predict one or more patient attributes and/or RTTPs to present to the Tumor Board member-users.

The analytics server can generate or train the machine learning language processing model. The analytics server can generate or train the machine learning language processing model using supervised learning, unsupervised learning, or semi-supervised learning techniques. For example, the analytics server can train the machine learning language processing model using a labeled training data set. The labeled training data set may include different sentences or paragraphs of text that correspond to the radiotherapy treatment of patients. The sentences or paragraphs of text may correspond to the radiotherapy treatment of patients, for example, because the sentences or paragraphs can include values of one or more patient attributes and may include specific keywords that correspond to radiotherapy treatment (e.g., radiotherapy, radiation, radiation therapy, oncology, tumor, radiation oncology, linear accelerator, radiation dose, external beam radiation), among others. In some cases, the labeled training data set can correspond to radiotherapy based on the source of the training data. For example, the labeled training data set can include sentences or text from clinical guidelines for radiotherapy treatment planning, case studies of radiotherapy treatment, medical journals and research papers on radiation therapy treatment, structured radiotherapy treatment plans, etc. The labeled training data set can include annotations indicating the rationale behind certain decisions in the treatment plans or any specific considerations that were taken into account. The labeled training data set can additionally or instead include labels indicating the correct responses to the different sentences or text. The analytics server can automatically label the training data set, or a human reviewer can label the training data set. Such text can be fed (e.g., by the analytics server) into the machine learning language processing model for training.

The analytics server can train the machine learning language processing model using backpropagation techniques. For example, the analytics server can insert different entries (e.g., prompts, such as sentences or text) in the training data set into the machine learning language processing model and execute the machine learning language processing model for each entry. In executing the model for an entry, the machine learning language processing model can generate or output a word or a sequence of words based on the words, images, and/or videos in the entry. The analytics server can determine a difference between the output of the machine learning language processing model and the label for the entry according to a loss function. The analytics server can use backpropagation techniques based on the difference to adjust the parameters and/or weights of the machine learning language processing model. The analytics server can train the machine learning language processing model in this manner over time with different labeled entries. The analytics server can train the machine learning language processing model until the machine learning language processing model is accurate to an accuracy threshold, at which point the analytics server can deploy the machine learning language processing model for use to collect patient attributes through the interaction interface.

During training, the analytics server may iteratively execute the machine learning language processing model to generate new predicted text, images, and/or videos based on the training dataset (e.g., for each entry of text, images, and/or videos). If the predicted results do not match the real outcome, the analytics server can continue the training unless and until the computer-generated recommendation satisfies one or more accuracy thresholds and is within an acceptable range. For instance, the analytics server may segment the training dataset into three groups (i.e., training, validation, and testing). The analytics server may train the machine learning language processing model based on the first group (training). The analytics server may then execute the (at least partially) trained machine learning language processing model to predict results for the second group of data (validation). The analytics server then verifies whether the prediction is correct. Using the above-described method, the analytics server may evaluate whether the machine learning language processing model is properly trained. The analytics server may continuously train and improve the machine learning language processing model using this method. The analytics server may then gauge the machine learning language processing model's accuracy (e.g., the area under the curve, precision, and recall) using the remaining data points within the training dataset (test).

At step 208, the analytics server can present on the interaction interface, the treatment attribute for the patient in the response. The response can be text requesting the second patient attribute of the patient. The second patient attribute that the machine learning language processing model determined would cause the data for the patient to satisfy a template condition of a template. The analytics server can present the response requesting the second patient attribute of the patient at the interaction interface being accessed by the user.

Referring again to FIG. 3, in a non-limiting example, the analytics server can present the response on the user interface 300. The response can be a request for a current body mass index of the patient. The analytics server can present the response at the interaction interface 302 of the user interface 300. The analytics server can present the response underneath the first input the user initially provided through the interaction interface 302.

In some cases, the analytics server can receive a second input from the user interface that includes a value of the second patient attribute requested in the response presented at the interaction interface. The user accessing the user interface can input the value (e.g., via text, an image, or a video) of the second attribute into the interaction interface. The response presented at the interaction interface can be text requesting the second patient attribute of the patient. The computing device presenting the user interface can transmit the value of the second patient attribute to the analytics server. The analytics server can receive the value as the second input.

The analytics server can receive the second input. The analytics server can execute the machine learning language processing model using the second input, the first input, and/or any retrieved patient data from memory to determine if any template conditions are satisfied. Responsive to determining there are not any satisfied template conditions, the machine learning language processing model can generate a request for another patient attribute (e.g., a third patient attribute). The analytics server can transmit the request for the third patient attribute to the computing device for display on a user interface. The user accessing the user interface can input a value for the third patient attribute and the computing device can transmit the value for the third patient attribute to the analytics server. The analytics server and the computing device presenting the user interface can repeat this process until the machine learning language processing model has received patient attributes to satisfy a plan condition.

A plan condition can be or include one or more rules that, upon satisfaction, indicate enough data or patient attributes have been collected to generate a radiotherapy treatment plan for a patient. A plan condition can be or include a template condition or any other set of rules.

Referring again to FIG. 3, in a non-limiting example, the analytics server can receive the second input containing the second patient attribute from the user interface 300. The user accessing the user interface 300 can input a value for the second patient attribute into the interaction interface 302. The second patient attribute can be, for example, potential treatment options for the patient. The user can input the value for the second patient attribute and values for any other patient attributes as a second input and select a submit button. The selection of the submit button can cause the computing device presenting the user interface 300 to transmit the second input to the analytics server.

In step 210, in response to receiving an indication of approval, transmitting, by the processor, the treatment attribute, the first input, and the second input to a radiation therapy plan optimizer as an instruction to generate a radiotherapy treatment plan for the patient. The radiotherapy plan optimizer can be a computer model (e.g., an optimization computer model) that is configured to generate one or more treatment attributes for a radiotherapy treatment plan that comply with the radiation therapy plan objectives for a patient (e.g., objectives in patient attributes of the patient) based on patient attributes of the patient for which the radiotherapy treatment plan is being generated. The radiotherapy plan optimizer can generate the one or more treatment attributes, for example, by iteratively calculating the one or more treatment attributes where, with each iteration, the radiotherapy plan optimizer revises the one or more attributes of the radiotherapy treatment plan in accordance with a cost value. The radiotherapy plan optimizer can receive the first patient attribute of the patient and the second patient attribute of the patient, in some cases in combination with other patient attributes that were used to satisfy a template or plan condition. Based on the patient attributes, the radiotherapy plan optimizer can generate or determine one or more attributes (e.g., radiation dose amounts or other information, field geometry settings, arc settings, treatment frequency, type of treatment, radiation parameters, etc.) of a radiotherapy treatment plan for the patient to generate a radiotherapy treatment plan.

The analytics server can generate a vector of tokens from the patient attributes. The analytics server can generate the vector of tokens using a tokenization algorithm, such as WordPiece or Byte Pair Encoding. In some cases, the analytics server can use the same tokenization algorithm that was used to train the radiotherapy plan optimizer to generate treatment attributes of a radiotherapy treatment plan. In generating the tokens, the analytics server can generate an array or vector of numbers from the patient attributes. In some cases, each attribute may correspond to a different number (e.g., map to a different number) and the analytics server can determine a number or token for each attribute based on the mappings. The analytics server can generate the vector of tokens from the patient attributes and transmit the vector of tokens to the radiotherapy plan optimizer.

In some cases, the analytics server can convert the vector of tokens to a structured data set. The structured dataset may include the data in the vector of tokens in a format that the radiotherapy plan optimizer can process. For example, the analytics server can convert the vector of tokens to a traditional structured cost function or field geometry definitions. For example, the analytics server can store a mapping of tokens to values of a cost function or field geometry settings. The analytics server can convert the vector of tokens to the traditional structure cost function or field geometry definitions based on the mapping. The analytics server can transmit the converted vector of tokens to the radiotherapy plan optimizer. In another example, the vector of tokens can contain a reference to one or more pre-defined treatment protocols (e.g., treatment protocol templates). Such tokens can be used to construct instructions to the radiotherapy plan optimizer. For example, in some cases, the token may instruct the plan optimizer to exclude and/or include certain treatment attributes from the radiotherapy treatment plan.

The radiotherapy plan optimizer can receive the patient attributes (e.g., the raw patient attributes, the vector of tokens representing the patient attributes, or the converted vector of tokens, as described above). A computer (e.g., the analytics server or another computer) storing the radiotherapy plan optimizer can execute the radiotherapy plan optimizer using the patient attributes as input. The radiotherapy plan optimizer can output one or more treatment attributes of a radiotherapy treatment plan for the patient based on the patient's attributes. The radiotherapy plan optimizer can transmit the one or more treatment attributes to the analytics server.

The analytics server can receive the one or more treatment attributes. The analytics server can present the radiotherapy treatment plan (e.g., the one or more treatment attributes of the radiotherapy treatment plan) on the user interface. The analytics server can present the radiotherapy treatment plan on the interaction interface of the user interface or on another portion of the user interface. In some embodiments, the analytics server can transmit the one or more treatment attributes to a radiotherapy treatment machine to use to provide treatment to the patient. The radiotherapy treatment machine can operate (e.g., automatically operate) based on the one or more treatment attributes.

In some cases, the user at the user interface may adjust the radiotherapy treatment plan. For example, the user viewing the radiotherapy treatment plan at the user interface can provide an input (e.g., a text input or a selection of a button) at the user interface that the radiotherapy treatment plan is incorrect. The analytics server can receive the input indicating the radiotherapy treatment plan is incorrect. Responsive to the input indicating the radiotherapy treatment plan is incorrect, the analytics server can execute the machine learning language processing model using the input or an indication of the input that the radiotherapy treatment plan is incorrect to generate a second response requesting a third patient attribute or a fourth patient attribute regarding the patient. The analytics server can present the second response requesting the third patient attribute or the fourth patient attribute of the patient at the interaction interface. The analytics server can receive, from the interaction interface, a third input comprising the requested third patient attribute or the fourth patient attribute of the patient. The analytics server can transmit the first patient attribute of the patient, the second patient attribute of the patient, and the third patient attribute or the fourth patient attribute of the patient to the radiotherapy plan optimizer (e.g., a vector of tokens or a converted vector of tokens generated based on the patient attributes). The radiotherapy plan optimizer can generate a second radiotherapy treatment plan for the patient based on the first patient attribute, the second patient attribute, and the third patient attribute or the fourth patient attribute. The radiotherapy plan optimizer can transmit the second radiotherapy treatment plan to the analytics server. The analytics server can present the second radiotherapy treatment plan at the user interface and/or transmit the second radiotherapy treatment plan to the radiotherapy machine.

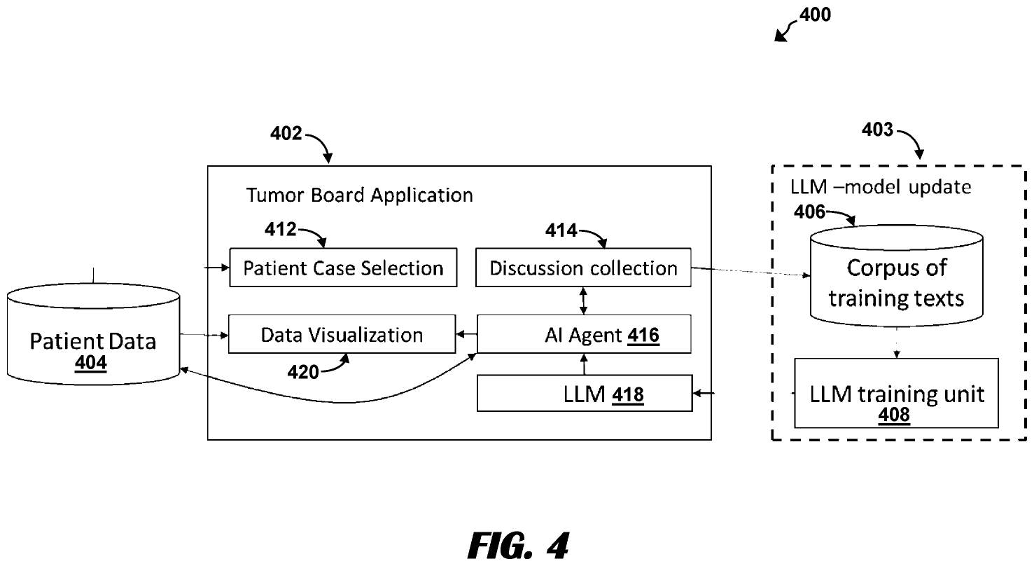

FIG. 4 illustrates a dataflow amongst components of a system 400 hosting a TBA 402 for Tumor Board collaboration and RTTP, according to an embodiment. The TBA 402 includes software programming executed by processors of computing devices (e.g., analytics server 110a of FIG. 1). The system 400 includes the TBA 402 programming, a patient database 404 (e.g., and an LLM-model learning subsystem 403, which includes a learning corpus database 406 and a training unit 408.

The TBA 402 includes various software modules having machine-executable software for execution by the processors of the computing devices. For ease of description and understanding, the example embodiment is described as having a single computing device having one processor that executes the TBA 402 and the learning subsystem 403.

The patient database 404 and the training database 406 may be hosted by any number of computing devices having hardware and software components, such as non-transitory storage memory, processors, and database management software (DBMS), and capable of performing the various tasks and processes described herein. For ease of description and understanding, the example embodiment includes a patient database 404 hosted on the same particular computing device as a training database 406, such the system database 110b of FIG. 1, though the patient database 404 and the training database 406 may be hosted on separate computing devices.

The computing device hosting the TBA 402 may host the patient database 404 or may communicate with the patient database 404 via one or more networks. The patient database 404 contains patient data relevant to the patient cases, such as medical care data records and multimedia (e.g., image scans). The separate LLM-model update subsystem 403 includes the training database 406 having persistent, non-transitory storage of prior and/or ongoing Tumor Board discussions, collected as inputs by the TBA 402. Programming of an LLM training unit 408 references the training database 406 to train new LLMs or update (retrain) LLMs, which may perform better in the tasks utilized in the TBA 402. The LLM training unit 408 retrieves training data entries of the training database 406 and applies the LLM model 418 on each training data entry. The LLM model 418 is trained to determine and statistically predict a next token or phrase for output based upon relationships in text or other data of the training data entries. For example, the training unit 408 starts with applying an untrained LLM model 418 (e.g., LLM model 418 having random or initialized weights or parameters) on a large corpus of training data, including sentences of text or other types of data from the training database 406 or other data sources. The LLM model 418 ingests the corpus of sentences in small chunks or tokens, and outputs a predicted next token or predicted output given a sequence of previous tokens. Optionally, in some embodiments, the training dataset may include labels indicating a correct, expected next token or a user may enter the correct, expected next token. The training unit 408 may otherwise have access to, parse, retrieve, or recognize the expected next token in the training data entry (e.g., sentence or phrase). The training unit 408 may receive or access the expected next token or expected output and perform a loss function that computes a loss between the predicted next token or predicted output compared against the expected next token or expected output.

As an example, given the sequence of tokens “consider radiotherapy treatment . . . ” then the LLM 418 might predict “guides” as the next word. The training unit 408 may determine from the training dataset entry that the expected next word is “plan,” and thus determine that the predicted next token (“guides”) is incorrect, rather than the expected next token (“plan”). The training unit 408 may compute the loss function and update or tune the weights or parameters of the LLM 418 to reduce the level of error and generate a better prediction in the next iteration of applying the LLM 418 on the next training data entry.

The TBA 402 can have the following software sub-components: a patient case selection engine (sometimes referred to as a patient case selector 412), a discussion collection and input ingestion engine (discussion collector 414), an AI agent 416, an LLM 418, and a data visualization 420. The case selector 412 determines which patient case is currently considered by the Tumor Board and retrieve the patient data from the patient database 404. The discussion collector 414 gathers and generates a discussion log for the Tumor Board discussions. The discussion collector 414 captures and records, to the discussion log, various spoken or written replies of the expert members of the Tumor Board. The AI agent 416 analyses the discussion log with the aid of the LLM model 418. The AI agent 416 may also interact with one or more other components of the TBA 402, such as the data visualization 420 for presenting data outputs produced by the AI agent 416 and LLM model 418.

The LLM training unit 408 may train and develop the LLM 418 through prompting and tooling processes, which are techniques for tuning more precise LLMs 418 for use in Tumor Board discussions. Prompting includes an approach to configuring the LLM 418 in which a user-operated input feed is embedded into a request template that specifies a problem statement in more detail for the LLM 418. This means, for example, adding additional instructions about which kind of result is expected, adding context that might be missing from the initial request, or giving a few examples of expected answers for given similar questions. The prompting functions include configuration instructions for response templates indicating, for example, the types of data to include in the responses or the structure of the responses, among other configurations for responses. The training unit 408, however, need not adjust the weights or parameters of the LLM 418 during prompting-based training functions. For instance, as part of the prompting functions, the prompt may instruct the LLM 418 to indicate that the LLM 418 or AI agent 416 should reference or invoke other software tools (e.g., a tool for performing certain calculations) outside of the LLM 418 generative programming methods when the LLM 418 or AI agent 416 constructs the outputted result.

Tooling is another approach to configuring the LLM 418 and outputs generated by the LLM 418. Tooling implements a combination of software tools that includes any number of software programs or other resources (e.g., training data) referenced and executed by the training unit 408 or other components. The tooling functions include the software components executed by the training unit 408 that may, for example, create, train, evaluate, and deploy the LLM 418. For instance, the training unit 408 may access or invoke the combination of software tools to develop, evaluate, and tune the LLM 418 to generate outputs related to the Tumor Board discussions. The software tools used for tooling includes, for example, various software programs, libraries, frameworks, and/or other computing resources that the training unit 408 may reference or execute to develop, train, fine-tune, and evaluate the predicted outputs of the LLM 418. The tooling functions executed by the training unit 408 may include, for example, collecting and preprocessing training data inputs, executing the LLM 418 during training, training or fine-tuning the LLM 418 weights or parameters, and executing a loss function (or other function for evaluating metrics and outputs of the LLM 418), among others. For instance, when performing the tooling functions, the training unit 408 (or other software components that train or tune the LLM 418) may invoke the various software tools to execute the tooling-based training operations, such as collecting training data from the training database 406 (or data sources) and/or preprocessing the training data, applying the LLM 418 on the training data, tuning the weights or parameters of the LLM 418 according to the loss function of the training unit 408 or another software tool.

The output feed of the TBA 402, as generated by the LLM 418 and AI agent 416, can then be augmented by the results of the external software tool. In some cases, the LLM's 418 task can then be repeated, now with the augmented feed in ongoing or future iterations of the LLM 418 and AI agent 416. As an example, complex math can be outsourced to a python prompt, or a search engine can be used to gain knowledge about recent events. In practice, the instructions of these tools are embedded via prompting to the feed and then the output feed is processed (using traditional search and pattern matching approaches) to extract the generated instructions for the external tool. The result of the external tool is then embedded back to the original input feed of the LLM 418. The LLM 418 operations (e.g., query using the search engine) may be repeated.

For a Tumor Board meeting, an end-user or a meeting software program automatically invokes or executes the TBA 402 to gather information during the Tumor Board meeting and generate outputs for the ongoing Tumor Board discussion. The discussion collector 414 captures the inputs of the experts participating in the Tumor Board meeting. In some implementations, the discussion collector 414 executes automatic speech recognition (ASR) software defining a machine-learning architecture and natural-language processing (NLP) engine that are trained to ingest audio signals, detect portions of the audio containing speech, and generate a text output of the spoken content, which the discussion collector 414 appends to the discussion log. Additionally or alternatively, one or more experts can input comments in written text via a user interface of the client devices of the experts. The discussion collector 414 may capture and append the written text inputs to the discussion log. The TBA 402 may add the written text entries of the discussion log into an LLM prompt interface to invoke the LLM 418.

The modules of the TBA (e.g., discussion collector 414) enters or otherwise inputs the written text of the discussion log into an LLM prompt interface. The TBA 400 provides a listing of known facts related to the particular patient case (in natural language or in structured form), and a listing of participants that indicates members of the Tumor Board participating in the prior or ongoing Tumor Board discussion. In addition, the TBA 400 introduces, invokes, or indicates the AI agent 416 and prepares an explanation or instruction of the contribution expected from the AI agent 416. The TBA 400 then adds the written text of the discussion log, so far, the end of the LLM prompt, so that the AI agent 416 and the LLM 418 will dynamically continue or advance the ongoing discussion. For instance, the LLM 418 may essentially predict or guess one or more outputs responsive to the context of the ongoing to discuss and provide a continuation of the discussion.

In some implementations, when a response text of the LLM 418 contains an instruction to use an additional software tool, the LLM 418, AI agent 416, or end-user may invoke the tool, which instructs a computing device hosting the software tool to launch the particular tool. The instructions to invoke or launch the software tool may include additional instructions generated by the LLM 418.

In some circumstances, the LLM 418 generates predicted text for continuation of the discussion amounts to a hallucination (e.g., LLM 418 predicts or detects which participant would speak next; predicts/detects what would be the next comment). The TBA 400 may omit such hallucinated text from outputs presented to the end-users (e.g., via a user interface of a monitor). However, if the LLM 418 predicts that next “speaker” is the AI agent 416, then the predicted comment of the AI agent 416 is processed (e.g., written down and presented via a user interface presented at a monitor; converted to audible speech signal).

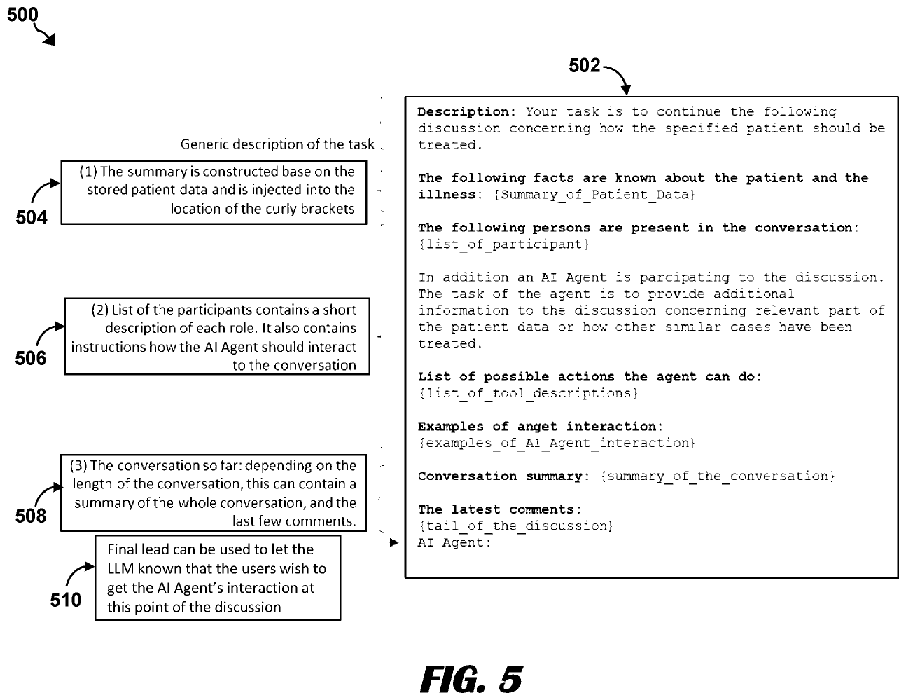

In some cases, the LLM prompt ends or concludes with a statement that the AI Agent 416 will be interacting next (e.g., agent output indictor 510 of FIG. 5), which forces the LLM 416 and LLM prompt to output an AI agent 416 action after calls to the LLM 418. And in some cases, another wording of the LLM prompt could indicate that the role of the LLM 418 is to generate, predict, or otherwise provide some kind of action for each reply of human expert. The list of possible actions generated by the LLM 418 can be inputted as part of the LLM prompt. In some cases, the LLM prompt can also provide examples how the TBA 400 should select the AI agent 418 action in different situations.

With reference to FIG. 5, which depicts an LLM prompt interface 500 displaying a template of an LLM prompt 502 to configure the LLM to generate predicted outputs for contributing to and continuing a discussion of a Tumor Board, according to an embodiment, according to an embodiment. The example LLM prompt 502 includes curly brackets that will be automatically populated or replaced by problem-specific content before the prompt 502 is fed to the LLM 418. The predicted or expected outcome of the LLM 418 is the most probable continuation of the discussion.

The modules of the TBA (e.g., discussion collector 414) input one or more written text entries of the discussion log into an LLM prompt 502. The TBA 400 starts by inputting a summary of patient facts 504, which may include a list or description containing known facts related to the patient case (in natural language or in structured text form), and a participant listing 506 containing indicators of the members of the Tumor Board participating with the prior or ongoing Tumor Board discussion. The participant listing 506 indicates that an AI agent 416 is a participant. The participant listing 506 (or other portion of the prompt 502) further includes instructions or expectations for how the AI agent 416 should function and an explanation of the contribution(s) expected from the AI agent 416. The discussion text 508 of the (whole or part of the) discussion log of the discussion, so far, is added to the end of the prompt 502 so that the LLM 418 via the AI Agent 416 may continue the discussion (by essentially trying to guess the continuation of the discussion). In some cases, the LLM prompt 502 includes an output indicator 510 that instructs or causes the LLM 418 and the AI agent 416 to provide the AI agent 416 interactions at a given point in the discussion.

With reference to FIG. 4, in some configurations, the LLM 418 may be instructed to describe additional or alternative types of actions than only natural language comments meant to be shown for the experts. The LLM prompt may include descriptions for types of actions or formats of outputs implemented by the LLM 418 and AI agent 416, such that the LLM 418 and AI agent 416 is instructed or configured to ingest inputs and/or produce outputs beyond only text-based natural language comments written by or intended for display to the participating experts. Non-limiting examples of the types of actions of the AI agent 416 include: displaying multimedia data (e.g., diagnostic images) or other forms of data (e.g., laboratory test results) to the Tumor Board members; querying the patient database 404 and identifying similar patient cases in the patient database 404; applying statistical models to evaluate expected outcomes based on the current information in the patient data or other data sources; preparing a discussion continuation plan or filling structured information describing the decisions made or RTTP generated by the Tumor Board; querying a database of current resources available in a clinic or care facility; and determining and displaying possible alternative timelines or locations for the patient treatment.

The system 400 may include multiple AI agents 416 stored in one or more databases, each trained to as a type of domain-specific or purpose-specific AI assistant. Each of the AI agents 416 may be trained for assisting a specific participant role that is needed in Tumor Board discussions. For example, a first AI assistant is trained to assist the role of radiologist, and a second AI assistant is trained to assist the role of oncologist.

The TBA 402 may run the LLM prompt at regular intervals, collecting the text of the discussion thus far at the given interval. Additionally or alternatively, the TBA 402 may run the LLM prompts in response to an inputted request received from one or more members of the Tumor Board. In some cases, the LLM prompt is run for the AI Agent interaction. For instance, when there is a pause in the discussion, or when one of the participants asks the AI Agent 416 to provide some information.

The TBA 402 may execute the model update subsystem 403. The model update subsystem 403 is configured to perform an automatic data-collection and LLM transfer-learning training loop. The text (or other data) of ongoing or prior discussions of the Tumor Boards may be stored into the training database 406, which the LLM training unit 408 references when training more-specialized or more accurate LLMs 418 or tuning existing LLMs 418. With more tuned LLMs 418, the ability of having more specialized AI agents 416 becomes more prominent (e.g., when the LLM 418 is trained with the prior text of enough prior discussions involving physicists and radiologists, then the LLM 418 and related AI agent 416 can also better anticipate the further comments of each of these specialists).

The AI agent 416 automatically presents the predicted output data (e.g., text, images, charts, medical records data) via the data visualization 420 or other user interface presented at the monitor(s) of the participants.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of this disclosure or the claims.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc., may be passed, forwarded, or transmitted via any suitable means including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module, which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein may be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What we claim is:

1. A method comprising:
presenting, by a processor, a user interface providing an interaction interface between a plurality of medical professionals communicating regarding a radiation therapy treatment of a patient during a tumor board review;
receiving, by the processor from the interaction interface, a first input comprising a first patient attribute of the patient and a second input corresponding to the radiation therapy treatment of the patient;
executing, by the processor, a machine learning language processing model using the first input and the second input to predict a treatment attribute for the patient, the machine learning language processing model configured to identify a second radiation therapy treatment for a second patient that corresponds to the first input and the second input based on a tumor board review for the second patient to predict the treatment attribute for the patient, wherein the machine learning language processing model is trained using a set of transcriptions of a set of tumor board reviews for a set of previously implemented radiation therapy treatments;
presenting, by the processor on the interaction interface, the treatment attribute for the patient; and
in response to receiving an indication of approval, transmitting, by the processor, the treatment attribute, the first input, and the second input to a radiation therapy plan optimizer as an instruction to generate a radiotherapy treatment plan for the patient.

2. The method of claim 1, wherein the treatment attribute for the patient is a timeline of radiation therapy treatment of the patient.

3. The method of claim 1, further comprising presenting, by the processor on the interaction interface, at least one of a medical image, laboratory data, or test result of the patient.

4. The method of claim 1, further comprising presenting, by the processor on the interaction interface, a hyperlink configured to direct the interaction interface to third-party data associated with the radiation therapy treatment.

5. The method of claim 1, wherein the machine learning language processing model is further trained using previously performed radiation therapy treatments.

6. The method of claim 1, further comprising in response to the second input satisfying a predetermined threshold, presenting, by the processor in the interaction interface, a warning message.

7. The method of claim 1, wherein the first input is a medical image.

8. A system, comprising:
a server comprising a processor and a non-transitory computer-readable medium containing instructions that when executed by the processor causes the processor to:
present a user interface providing an interaction interface between a plurality of medical professionals communicating regarding a radiation therapy treatment of a patient during a tumor board review;
receive, from the interaction interface, a first input comprising a first patient attribute of the patient and a second input corresponding to the radiation therapy treatment of the patient;
execute a machine learning language processing model using the first input and the second input to predict a treatment attribute for the patient, the machine learning language processing model configured to identify a second radiation therapy treatment for a second patient that corresponds to the first input and the second input based on a tumor board review for the second patient to predict the treatment attribute for the patient, wherein the machine learning language processing model is trained using a set of transcriptions of a set of tumor board reviews for a set of previously implemented radiation therapy treatments;
present, on the interaction interface, the treatment attribute for the patient; and
in response to receiving an indication of approval, transmit the treatment attribute, the first input, and the second input to a radiation therapy plan optimizer as an instruction to generate a radiotherapy treatment plan for the patient.

9. The system of claim 8, wherein the treatment attribute for the patient is a timeline of radiation therapy treatment of the patient.

10. The system of claim 8, wherein the instructions further cause the processor to present, on the interaction interface, at least one of a medical image, laboratory data, or test result of the patient.

11. The system of claim 8, wherein the instructions further cause the processor to present, on the interaction interface, a hyperlink configured to direct the interaction interface to third-party data associated with the radiation therapy treatment.

12. The system of claim 8, wherein the machine learning language processing model is further trained using previously performed radiation therapy treatments.

13. The system of claim 8, wherein the instructions further cause the processor to, in response to the second input satisfying a predetermined threshold, presenting, by the processor in the interaction interface, a warning message.

14. The system of claim 8, wherein the first input is a medical image.

15. A system, comprising:
a computer configured to display a user interface; and
a server in communication with the computer, the server configured to:
present the user interface providing an interaction interface between a plurality of medical professionals communicating regarding a radiation therapy treatment of a patient during a tumor board review;
receive, from the interaction interface, a first input comprising a first patient attribute of the patient and a second input corresponding to the radiation therapy treatment of the patient;
execute a machine learning language processing model using the first input and the second input to predict a treatment attribute for the patient, the machine learning language processing model configured to identify a second radiation therapy treatment for a second patient that corresponds to the first input and the second input based on a tumor board review for the second patient to predict the treatment attribute for the patient, wherein the machine learning language processing model is trained using a set of transcriptions of a set of tumor board reviews for a set of previously implemented radiation therapy treatments;
present, on the interaction interface, the treatment attribute for the patient; and
in response to receiving an indication of approval, transmit the treatment attribute, the first input, and the second input to a radiation therapy plan optimizer as an instruction to generate a radiotherapy treatment plan for the patient.

16. The system of claim 15, wherein the treatment attribute for the patient is a timeline of radiation therapy treatment of the patient.

17. The system of claim 15, wherein the server is further configured to present, on the interaction interface, at least one of a medical image, laboratory data, or test result of the patient.

18. The system of claim 15, wherein the server is further configured to present, on the interaction interface, a hyperlink configured to direct the interaction interface to third-party data associated with the radiation therapy treatment.

19. The system of claim 15, wherein the machine learning language processing model is further trained using previously performed radiation therapy treatments.

20. The system of claim 15, wherein the server is further configured to, in response to the second input satisfying a predetermined threshold, presenting, by the processor in the interaction interface, a warning message.
